(19) United States
(12) Patent Application Publication
(10) Pub. No.: US 2020/0023155 A1
(43) Pub. Date: Jan. 23, 2020

(54) ELECTROLYTIC HYDROGEN AND OXYGEN GAS INHALER

(71) Applicant: Takashi TAKEHARA, Osaka (JP)

(72) Inventor: Takashi TAKEHARA, Osaka (JP)

(21) Appl. No.: 16/484,208

(22) PCT Filed: Feb. 19, 2018
(86) PCT No.: PCT/JP2018/005632

§ 371 Date: Aug. 7, 2019

(30) Foreign Application Priority Data
Feb. 20, 2017 (JP) 2017-029446

(51) Int. Cl. A61M 16/10; C25B 15/08; C25B 1/04

(52) U.S. Cl. CPC A61M 16/1005; A61M 2016/0024; C25B 1/04; C25B 15/08

(57) ABSTRACT

Provided is a portable electrolytic hydrogen and oxygen gas suction tool capable of selectively generating hydrogen and oxygen. This electrolytic hydrogen and oxygen gas suction tool is characterized by including an electrolysis tank capable of storing water and constituted by an upper part and a lower part which are fluidically connected to each other therein and integrally molded, a pair of electrodes disposed in the lower part in the electrolysis tank, standing substantially in parallel with a vertical direction of the electrolysis tank, and faced with each other in a lateral direction, a battery, and a control substrate which supplies power from the battery, in which the electrode is supplied with or shut off from power supply from the battery by the control substrate.

### TECHNICAL FIELD

The present invention relates to an electrolytic hydrogen and oxygen gas suction tool which is portable and can easily supply predetermined amounts of hydrogen gas or oxygen gas selectively.

### BACKGROUND ART

Active oxygen has extremely strong oxidizability and plays a role of removing bacteria and viruses having entered the human body, but a research says that it attacks and even damages normal cells of a human. Presence of excessive active oxygen increases a possibility of damaging the normal cells and incurs risks of deterioration and mutation of cells or aging of the skin with that.

Recently, a research has discovered that hydrogen removes active oxygen, and it has attracted an attention since it is effective for health and beauty. In a reaction between hydrogen and active oxygen, only water is generated as a reactant and thus, a bad influence on the human body is extremely small. Therefore, intake of hydrogen into the body is recommended particularly for preventing aging or for promoting beauty/health in various states during physical exercises, eating and drinking, smoking, stay under ultraviolet/contaminated environments, and under a high stress such as lack of sleep and long-hour work, in which active oxygen tends to be generated easily in the body.

Moreover, oxygen is used for generating energy of a cell and is an indispensable element for metabolism of the human body. Attention has been paid to activation of the cells in the body by oxygen, and studies have been made in recent years that conscious intake of oxygen into the body is effective in promotion of natural healing of disease conditions such as fatigue recovery and fracture, improvement of hematogenous disorder, beauty, stress reduction and the like. Actually, it is known that athletes use oxygen capsules for body shaping or treatment of injuries, and oxygen masks are used for patients with weakened physical strength.

In addition to the attention to the intake of hydrogen and oxygen into the body as described above, moreover, in view of pseudo electronic cigarettes in the recent non-smoking boom or an expansion of the market for cigarettes not emitting sidestream smoke, hidden needs for smoking hydrogen or oxygen leading to health promotion is considered to be large.

As a method of generating hydrogen and oxygen, a method of electrolyzing water is generally known. This is a method of breaking down water (H₂O) into hydrogen (H₂) and oxygen (O₂) by immersing an electrode in an aqueous solution and electrically conducting it, and only hydrogen and oxygen can be obtained without generating other harmful substances and the like by using tap water which can be easily obtained and handled. For example, Patent Literature 1 discloses a desktop hydrogen generating device which can generate hydrogen and oxygen without mixing by putting water in an electrolysis tank including an electrolytic plate in which a pair of electrode plates are brought into close contact on both surfaces of an ion exchange membrane and by electrically conducting it. Since this hydrogen generating device can be used by being arbitrarily moved by a user, convenience in handling is improved as compared with the hydrogen generating device which can be used only in an installed form.

However, though a size of the aforementioned desktop hydrogen generating device was reduced to some degree, it has not achieved such size that is suitable to be carried by the user in a bag or the like, and a power supply from an outlet needs to be ensured for use, and its moving range was limited for utilizing it as a hydrogen and oxygen gas suction tool for suctioning into the body by the user. Moreover, it was the device for obtaining only hydrogen, which does not assume intake of oxygen by the user, and the need for intake of only hydrogen, only oxygen or selectively both in accordance with the health state or use purpose of the user has not been responded.

Moreover, assuming an instrument of a portable size, disposition of an ion exchange membrane which is a separate member and a separate material in a small-sized instrument requires precise work and design, and a request for cost reduction for general use could not be satisfied, either.

### CITATION LIST

### Patent Literature

[Patent Literature 1] Japanese Patent Laid-Open No. 2014-019640

### SUMMARY OF INVENTION

### Technical Problem

The present invention was made in view of the aforementioned circumstances and has an object to provide an electrolytic hydrogen and oxygen gas suction tool which is of a charging type, small-sized and inexpensive so that a user can freely carry it and moreover, capable of selectively generating hydrogen and oxygen.

### Solution to Problem

In order to solve the aforementioned problem, an electrolytic hydrogen and oxygen gas suction tool of the present invention includes:

an electrolysis tank capable of storing water and constituted by an upper part and a lower part which are fluidically connected to each other therein and integrally molded;

a pair of electrodes disposed in the lower part in the electrolysis tank, standing substantially in parallel with a vertical direction of the electrolysis tank, and faced with each other in a lateral direction;

a battery; and

a control substrate which supplies power from the battery, in which

the electrode is supplied with or shut off from power supply from the battery by the control substrate;

a partition member extending downward from a boundary between the upper part and the lower part of the electrolysis tank passing between the pair of electrodes is integrally molded and provided in the lower part of the electrolysis tank;

the pair of electrodes are fluidically connected to each other in the lower part of the electrolysis tank; and

opening/closing means which enables switching of gaseous connection between one and/or the other of the pair of electrodes separated by the partition member and the upper part of the electrolysis tank is provided.

According to the aforementioned electrolytic hydrogen and oxygen gas suction tool, first, the pair of electrodes disposed in the electrolysis tank are electrically conducted by the control substrate, hydrogen is generated by electrolysis of the water in the vicinity of one of the electrodes (negative electrode) in the electrolysis tank, and oxygen is generated in the vicinity of the other electrode (positive electrode). And mixing of air bubbles of hydrogen and oxygen is inhibited by the partition member extending between the pair of electrodes, and moreover, movement of the hydrogen or oxygen to the upper part in the electrolysis tank is controlled by the opening/closing means so that the gas can be discharged to an outside of the device through the upper part of the electrolysis tank in an “open” state, while the gas can be stored in the lower part of the electrolysis tank in a “closed” state. By means of this opening/closing means, the hydrogen or oxygen generated in the electrode can be selectively obtained. Moreover, since the present invention has simple configuration of the integrally molded electrolysis tank, electrodes, battery and control plate and the like, the hydrogen and oxygen gas suction tool can be made inexpensive and in a portable size.

Moreover, in the electrolytic hydrogen and oxygen gas suction tool of the present invention, in the lower part of the electrolysis tank and above the pair of electrodes, it is preferable that the partition member is formed by a plate member, and passage of a fluid and the gas is shut off between a one surface side and the other surface side of the partition member.

According to the aforementioned electrolytic hydrogen and oxygen gas suction tool, in the hydrogen and oxygen generated in the vicinity of the electrodes, by considering the air bubbles moving upward in the aqueous solution, mixing of the air bubbles of the hydrogen and oxygen through the aqueous solution above the electrodes can be further prevented.

The opening/closing means is a member provided on the boundary between the upper part and the lower part of the electrolysis tank and having a closed and substantially flat area and may be characterized by that the substantially flat area moves on a substantial plane in parallel with the boundary between the upper part and the lower part of the electrolysis tank in accordance with an operation by the user.

According to the aforementioned electrolytic hydrogen and oxygen gas suction tool, by parallel movement of the opening/closing means on the plane, an opening formed on the boundary between the upper part and the lower part of the electrolysis tank is selectively opened/closed. That is, with a simple mechanism in which the member is moved on the plane, resistance of the water hardly affects, easy opening/closing by a manual force which is easy for the user in the manual case, and a dynamic load on the instruments is small and thus, durability of the members can be improved, and costs of the members can be kept low.

Moreover, in the electrolytic hydrogen and oxygen gas suction tool of the present invention, the battery is disposed in parallel in the vertical direction of the electrolysis tank, and above the battery, an aromatic gas generating member in which on/off control of the aromatic gas generation is conducted by the control plate is disposed, and a channel in which the aromatic gas is merged with the gas emitted from the electrolysis tank may be provided.

According to the aforementioned electrolytic hydrogen and oxygen gas suction tool, by disposing the battery and the aromatic gas generating member in parallel with the electrolysis tank, portability is improved by a shape which is small-sized and easy to be carried, and the user can selectively intake hydrogen and oxygen. Moreover, since hydrogen or oxygen with aroma can be enjoyed in accordance with preference of the user, a product can be provided to which a user who uses an existing electronic cigarette with aroma can change without a sense of discomfort and further as a product having a health enhancing function.

Moreover, in the electrolytic hydrogen and oxygen gas suction tool of the present invention, the opening/closing means may be controlled by the control plate.

According to the aforementioned electrolytic hydrogen and oxygen gas suction tool, the opening/closing means can be operated by sending an operation signal to the control plate not with a manual cumbersome operation but with a simple operation such as touching, and hydrogen and oxygen can be selectively generated easily.

Moreover, another electrolytic hydrogen and oxygen gas suction tool of the present invention includes:

an electrolysis tank capable of storing water and constituted by an upper part and a lower part which are fluidically connected to each other therein and integrally molded;

a pair of electrodes disposed in the lower part in the electrolysis tank, standing substantially in parallel with a vertical direction of the electrolysis tank, and faced with each other in a lateral direction;

a battery; and

a control substrate which supplies power from the battery, in which

the electrode is supplied with or shut off from power supply from the battery by the control substrate;

a partition member extending downward from a boundary between the upper part and the lower part of the electrolysis tank passing between the pair of electrodes is integrally molded and provided in the lower part of the electrolysis tank; the pair of electrodes are fluidically connected to each other in the lower part of the electrolysis tank; gaseous connection between the one side of the pair of electrodes separated by the partition member and the upper part of the electrolysis tank is shut off, and the gaseous connection between the other side of the electrode and the upper part of the electrolysis tank is opened; and polarity inverting means which inverts polarity of power supplied to each of the pair of electrodes is provided.

In this electrolytic hydrogen and oxygen gas suction tool, in the aforementioned electrolytic hydrogen and oxygen gas suction tool of the present invention, it is configured such that the hydrogen or oxygen generated in the electrode by the opening/closing means can be selectively obtained, but in the aforementioned another electrolytic hydrogen and oxygen gas suction tool, hydrogen or oxygen can be selectively obtained by inverting the polarity of power to each of the electrodes as a method of selective obtainment and by closing an upper part on one electrode side so that the gas emitted to above the electrolysis tank is either hydrogen or oxygen. According to this method, hydrogen or oxygen can be selectively obtained electrically only by providing a polarity inversion circuit (polarity inverting means) on the control substrate or a separate power supply circuit.

Moreover, such a case can be considered specifically that the polarity inverting means has a polarity circuit which switches the polarity of power supplied from the battery each time an alternate-type switch is turned ON.

The “alternate” type switch is a type in which even the hand leaves a button after pressing it, the ON state is kept, and in this case, once the button is pressed to get ON, hydrogen or oxygen is continuously emitted as it is, and by pressing the button again, oxygen can be made to emit.

As another example of the polarity inverting means, provision of a polarity circuit which switches the polarity of power supplied from the battery by turning ON-OFF-ON the switch can be considered.

In the case of this polarity inverting means, there is no need to provide a separate power OFF switch, and emission of hydrogen at the first ON, oxygen at the second ON, and hydrogen again at the third ON, for example, can be performed. In the case of this example, the switch may be a momentary type, and hydrogen or oxygen can be emitted only while the button is pressed.

The polarity inverting means which inverts the polarity of the power supplied to each of the pair of electrodes can be also utilized for the present invention which can selectively obtain hydrogen or oxygen by using the closing means.

That is because, when emission/stop of hydrogen/oxygen is to be performed in a short time, stop is realized at once by operating the polarity inversion circuit. Therefore, it is advantageous when intake amounts or intake time of hydrogen/oxygen are to be controlled precisely.

### Advantageous Effect of Invention

According to the present invention, the electrolytic hydrogen and oxygen gas suction tool which can be carried freely by the user and capable of selectively obtaining hydrogen or oxygen can be provided. According to this electrolytic hydrogen and oxygen gas suction tool, the user can easily select hydrogen and oxygen (or both at the same time) in accordance with a health state and a use purpose of the user and take into the body regardless of the place.

### BRIEF DESCRIPTION OF DRAWINGS

FIG. 1 is an assembling/disassembling diagram exemplifying each member of an electrolytic hydrogen and oxygen gas suction tool of the present invention.

FIG. 2 illustrate views of the electrolytic hydrogen and oxygen gas suction tool of the present invention in FIG. 1 when seen from each direction, in which FIG. 2(a) is a left side view, FIG. 2(b)is a front view, FIG. 2(c)is a right side view, FIG. 2(d) is a bottom view, and FIG. 2(e) is a top view.

FIG. 3 illustrates a sectional view of the electrolytic hydrogen and oxygen gas suction tool of the present invention in FIGS. 1 to 2 along a line A-A in FIG. 2(c).

FIG. 4 is a view illustrating a lower part of an electrolysis tank of the electrolytic hydrogen and oxygen gas suction tool of the present invention and illustrates the lower part of the electrolysis tank in the view corresponding to a sectional view along a line B-B in FIG. 2(b).

FIGS. 5(a) and 5(b) both illustrate schematic views of movement of hydrogen 2 5 and oxygen and the like in FIG. 4.

FIGS. 6(a) and 6(b) both illustrate schematic views examples of movement of hydrogen and oxygen in FIG. 4 when another partition member is employed.

FIGS. 7(a), 7(b), and 7(c) all illustrate another embodiment of the partition member in the electrolytic hydrogen and oxygen gas suction tool of the present invention.

FIG. 8(a) illustrates a top view of an opening in the lower part of the electrolysis tank in another example of the electrolytic hydrogen and oxygen gas suction tool of the present invention, FIG. 8(b) illustrates a top view of opening/closing means, FIG. 8(c) illustrates top view of an example in which (b) is mounted on (a).

FIG. 9(a) illustrates a top view of an opening in the lower part of the electrolysis tank in another example of the electrolytic hydrogen and oxygen gas suction tool of the present invention, FIG. 9(b) illustrates a top view of opening/closing means, FIG. 9(c) illustrates top view of an example in which (b) is mounted on (a).

FIGS. 10(a) and 10(b) illustrate top views of the opening/closing means and FIGS. 10(c) and 10(d) illustrate top views of the opening in the lower part of the electrolysis tank in another example of the electrolytic hydrogen and oxygen gas suction tool of the present invention.

FIGS. 11(a) and 11(b) illustrate the embodiments of the hydrogen and oxygen gas suction tool of the present second invention as a modification in FIGS. 4 to 5.

FIGS. 12(a) and (b) illustrate an example of polarity inverting means (polarity inversion circuit), in which FIG. 12(a) illustrates a state before polarity inversion and FIG. 12(b) illustrates a state after the polarity inversion.

FIG. 13 illustrates another example of the polarity inverting means (polarity inversion circuit).

### DESCRIPTION OF EMBODIMENTS

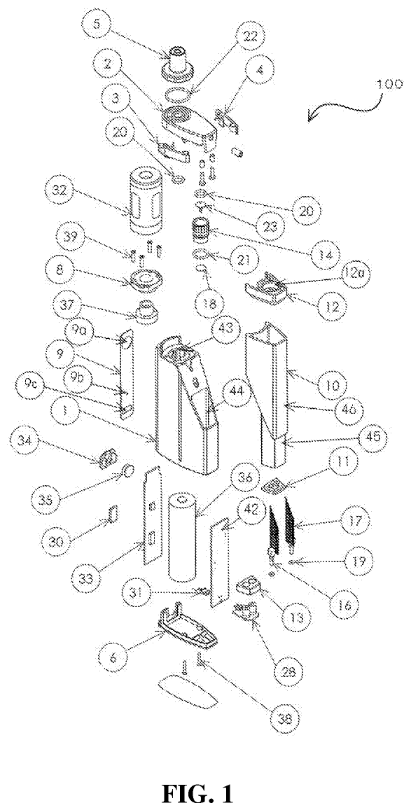

Hereinafter, typical examples of embodiments of the electrolytic hydrogen and oxygen gas suction tool of the present invention will be described in detail by referring to FIGS. 1 to 6. The electrolytic hydrogen and oxygen gas suction tool of the present invention is not limited to the illustrated ones but it is needless to say that modifications of the contents of the illustration and description within a range of common sense are also included. Moreover, each figure illustrates a dimension, a ratio or a number in an exaggerated way as necessary for easy understanding in some cases.

As described above, the electrolytic hydrogen and oxygen gas suction tool of the present invention is characterized by including a partition member and opening/closing means which are main configurations for separating generated hydrogen and oxygen.

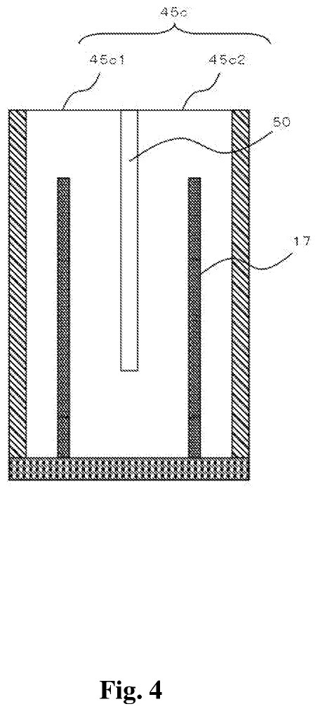

In describing the present invention, for simple understanding, first, the configuration “except the partition member and the opening/closing means” will be described in detail by using FIGS. 1 to 3 and then, the “configuration of the partition member and the opening/closing means” will be described in detail by using FIGS. 4 to 6.

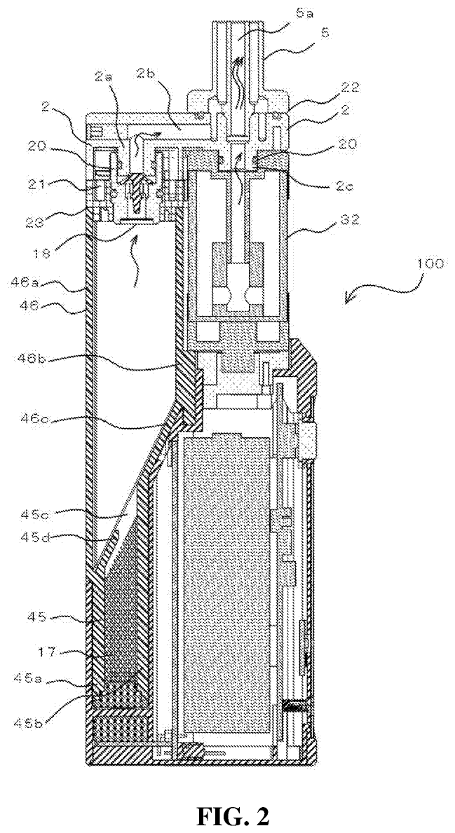

FIG. 1 is an assembling/disassembling diagram exemplifying each member of an electrolytic hydrogen and oxygen gas suction tool 100 of the present invention. FIGS. 2 illustrate views of the electrolytic hydrogen and oxygen gas suction tool 100 of the present invention when seen from each direction, in which FIG. 2(a) illustrates a left side view, FIG. 2(b) illustrates a front view, FIG. 2(c) illustrates a right side view, FIG. 2(d) illustrates a bottom view, and FIG. 2(e) illustrates a top view. In this description, an up-and-down direction and a vertical direction refer to an up-and-down direction on the drawing and a vertical direction on the drawing in (b), while a width direction, a lateral direction, and a side portion side refer to a right-and-left direction on the drawing, a lateral direction on the drawing, and right-and-left side portion sides on the drawing in (b).

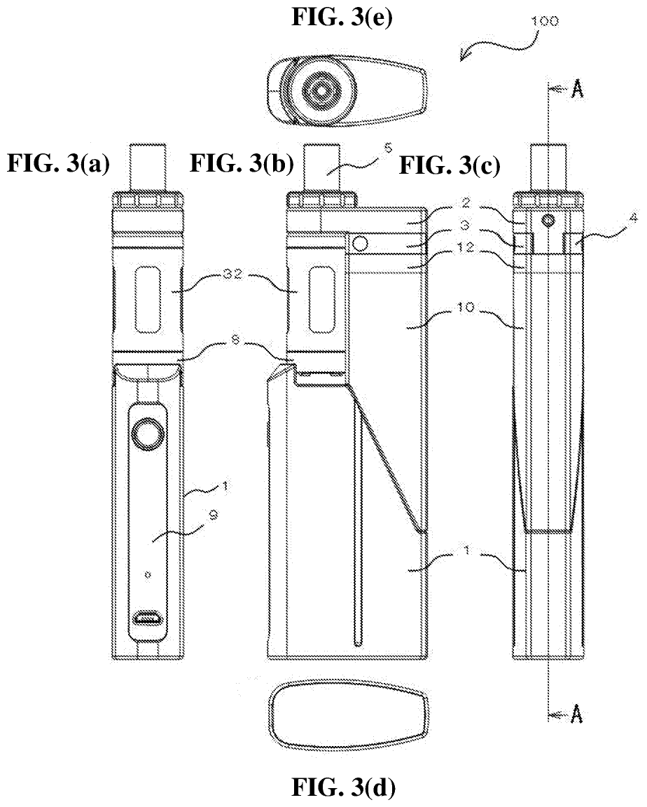

Moreover, FIG. 3 illustrates a sectional view of the electrolytic hydrogen and oxygen gas suction tool 100 in FIGS. 1 to 2 along a line A-A in FIG. 2(c).

Hereinafter, the electrolytic hydrogen and oxygen gas suction tool 100 will be described referring mainly to the assembling/disassembling diagram in FIG. 1, and the other figures will be referred to for convenience of the description.

As described above, FIG. 1 illustrates a configuration diagram of each member in this hydrogen and oxygen gas suction tool 100. A body cover 1 is a case made of a resin in which a battery receiving portion 43 opened upward and into which an entire battery 36 is inserted/stored in the vertical direction from the opening and an electrolysis tank receiving portion 44 having a shape arranged in parallel in the vertical direction with the battery receiving portion 43 and into which a reduced diameter portion 45 on a lower part of the electrolysis tank 10 can be inserted and fitted from above are provided. A battery 36 used here is preferably a charging-type lithium battery.

The body cover 1 has a shape which is longer on the battery receiving portion 43 side, and the electrolysis tank receiving portion 44 side is cut out so that the upper part is inclined to the side. A bottom portion of the body cover 1 can open/close the bottom portion of the battery receiving portion 43 with a body bottom cover 6 as a lid member and closes the bottom portion of the battery receiving portion 43 with the body bottom cover 6 after the battery 36 is inserted from the bottom portion during assembling. The body bottom cover 6 is closed by cross recessed screws 38. Moreover, in the body cover 1, spaces in which two control substrates (electronic substrates) 33 and 42 are disposed so as to sandwich the battery 36 in the vertical direction on the both side portion sides of the battery receiving portion 43 are provided, and the control substrate 33 on the side surface side of the body cover 1 is a main control substrate and controls power supply from the battery 36 with the control substrate 42 on the electrolysis tank 10 side performing power supply to a suctioning portion 32 (aroma generating device) and a mesh electrode 17 (electrode plate).

A decorative laminated sheet 9 is attached to the side surface of the body cover 1 along the longitudinal side surface, and a button hole 9a through which an operation button 35 to the control substrate 33 is seen, a hole 9b for LED for light irradiation from an LED substrate 30, and a hole 9c for charging connector for connecting a connector for charging the battery 36 from an external power source are provided.

By pressing on the operation button 35 three times, a power supply signal is transmitted in the control substrate 33 to the control substrate 42, and power of the battery 36 is supplied from the control substrate 42 for a predetermined time to a pair of the mesh electrodes (electrode plates) 17 through a housing 31 for substrate connector and a crimping substrate 28. When the power is supplied to the mesh electrode 17, the power supply signal is transmitted in the control substrate 33 to the LED substrate 30, and the LED substrate 30 causes the LED to emit light. As a result, the user can visually recognize a hydrogen and oxygen gas generation state by the hole 9b for LED. Pressing on the operation button 35 three times was made a condition for the power supply to the mesh electrode 17 as a safety condition to avoid unintentional button operation and power supply when the user moves with this hydrogen and oxygen gas suction tool 100 put in a pocket or the like.

The mesh electrodes 17 are disposed upward in a pair of two electrodes in parallel longitudinally, each forming positive/negative electrodes and corresponding to power from the positive/negative poles of the battery 36. Moreover, an upper end of the mesh electrode 17 has a shape cut out diagonally so as to correspond to a boundary line between the reduced diameter portion 45 and a water storage body portion 46 of the electrolysis tank 10. To a lower end of the mesh electrode 17, a rod-shaped titanium electrode 16 is connected so as to stand upright on a terminal substrate 28 and can be electrically connected thereto. In order to shield the mesh electrode 17 and the terminal substrate 28 from water in a state where the mesh electrode 17 stands upright, a packing 13 (made of a resin such as silicone) to be attached on the terminal substrate 28 and an O-ring (made of resin such as silicone: hereinafter the same applies to the O-ring) attached around the titanium electrode 16 are provided.

The electrolysis tank 10 is a container for storing water, the reduced diameter portion 45 and the water storage body portion 46 are integrally formed in order from below, and they are fluidically connected to each other therein. The water storage body portion 46 is opened upward so that water can be poured in and is half-closed by attaching an electrolysis tank lid 12. The electrolysis tank lid 12 penetrates vertically, and a penetrating opening 12a for receiving an umbrella valve 23, a screw cap 14 and the like is provided. In the water storage body portion 46, an outer side portion 46a forms a side wall substantially flat in the lateral direction from an upper end to a lower end as illustrated in FIG. 3 and is connected as it is to the upper end of the reduced diameter portion 45, and an inner side portion 46b on the body cover 1 side has a bottom portion 46c formed in parallel with the outer side portion 46a from the upper end to a lower center position and bending from the lower center position and inclined. The bottom portion 46c extends to a middle position in the lateral direction and is connected to the upper end of the reduced diameter portion 45.

Moreover, the reduced diameter portion 45 is thinner than the water storage body portion 46 as described above, and the upper end of the outer side portion 46a on the side wall side is continuously connected as it is to the lower end of the outer side portion 46a of the water storage body portion 46 and extends to the lower end as illustrated in FIG. 3, and the upper end of the inner side portion 45b on the body cover 1 side is bent downward and connected at a position of a distal end (edge part) of the bottom portion 46c of the water storage body portion 46 and extends to the lower end in parallel with the inner side portion 45b.

Moreover, at a connection position between the lower end of the outer side portion 46a of the water storage body portion 46 and the upper end of the outer side portion 46a of the reduced diameter portion 45, a water shielding plate 45d inclined substantially the same as the bottom portion 46c of the water storage body portion 46 and extending to an opening 45c is provided. This water shielding plate 45d extends inside the whole region in a perpendicular direction on the drawing in FIG. 3. Therefore, even if an aqueous solution collecting in the electrolysis tank 10 is electrolyzed and a water storage amount decreases, the water is stored substantially in the whole region inside the reduced diameter portion 45 at all times. More specifically, when the water storage amount decreases and an air layer is formed partially in the electrolysis tank 10, since the reduced diameter portion 45 is thinner than the water storage body portion 46, water is fully filled in the reduced diameter portion 45 in a normal standing state, and the air layer is not generated unless the water storage amount decreases too much.

Moreover, when the water storage amount decreases to some degree, too, the air layer can be generated in the reduced diameter portion 45 if this hydrogen and oxygen gas suction tool 100 is inclined or placed horizontally, but in the case of this electrolysis tank 10, water is fully filled in the reduced diameter portion 45 even in this case. More specifically, in the case of inclination in the left direction on the drawing in FIG. 3, for example, the bottom portion 46c becomes a baffle plate, and the air layer is formed on the inner side portion 46b side in the water storage body portion 46. On the other hand, in the case of inclination in the right direction on the drawing in FIG. 3, the water shielding plate 45d becomes the baffle plate, and the air layer is formed only on the outer side portion 46a side of the water storage body portion 46. Therefore, the entire mesh electrode 17 disposed in the reduced diameter portion 45 is in contact with water at all times, and even if the user is suctioning sideways, amounts of hydrogen and oxygen generated can be ensured at all times.

An upper end edge of the mesh electrode 17 is formed by being cut out diagonally so that the electrode is immersed in water in the reduced diameter portion 45 without a gap by following the shapes of the reduced diameter portion 45 and the opening 45c. Returning to FIG. 1 again, the lower end of the electrolysis tank 10 is closed by an electrolysis tank bottom 11, but the electrolysis tank bottom 11 has a pair of through holes into which the mesh electrodes 17 are inserted provided, and when the reduced diameter portion 45 of the electrolysis tank 10 is inserted into the electrolysis tank receiving portion 44 of the body cover 1, the mesh electrode 17 passes through the through hole of the electrolysis tank bottom 11 and is positioned in the reduced diameter portion 45.

The umbrella valve 23 and the like attached to the penetrating opening 12a of the electrolysis tank lid 12 on the upper end of the electrolysis tank 10 will be described. To the penetrating opening 12a, the screw cap 14 having an opening on an upper part and penetrating vertically is attached, and at that time, a vent filter 18 is interposed between a hole in the bottom portion of the screw cap 14 and the bottom portion of the penetrating opening 12a, and the O-ring 21 is inserted into the periphery on a lower part of the screw cap 14. The vent filter 18 is a micro hole and has a function of preventing water/dusts while adjusting an internal pressure in the opening of the screw cap 14. Moreover, the O-ring 21 shields a space between an outer peripheral wall of the opening in the screw cap 14 and an inner peripheral wall of the penetrating opening 12a from water.

Moreover, the umbrella valve 23 (made of a material having flexibility such as silicone) operating in the up-and-down direction is attached in the opening of the screw cap 14, and when the user suctions through the nozzle 5 (which will be described later), and a negative pressure acts upward, the umbrella valve 23 is raised/operated and is fluidically connected to the inside of the electrolysis tank 10 through a through hole in the bottom portion of the screw cap 14 and the penetrating opening 12a of the electrolysis tank lid 12. Therefore, when the user suctions through the nozzle 5, the hydrogen or oxygen gas raised and collecting in the electrolysis tank 10 is emitted to an outside. On the contrary, if the user stops suctioning, and a state in which the negative pressure does not act is brought about, the umbrella valve 23 is lowered/operated, the through hole in the bottom portion of the screw cap 14 is closed, and the emission of the hydrogen or oxygen gas in the electrolysis tank 10 is closed.

To the electrolysis tank lid 12 to which the screw cap 14 and the umbrella valve 23 are attached, a mixer 2 is attached from above. The mixer 2 has a cylindrical member 2a extending downward as illustrated in FIG. 3, and by inserting a lower end of the cylindrical member 2a into the opening of the screw cap 14, the cylindrical member 2a forms a channel for guiding the hydrogen or oxygen gas from the umbrella valve 23 upward. The O-ring 20 is provided around the outer peripheral wall of this cylindrical member 2a and seals a gap from the inner wall of the opening in the screw cap 14.

Fixation of the mixer 2 and the electrolysis tank lid 12 is accomplished by attaching lock buttons 3 and 4. The lock buttons 3 and 4 are sandwiched and snap-fastened in a front-and-rear direction (the perpendicular direction on the drawing in FIG. 3) at a gap position in the up-and-down direction between the mixer 2 and the electrolysis tank lid 12, respectively. Moreover, the mixer 2 has a channel 2b provided toward the nozzle 5 direction in an upper part thereof as illustrated in FIG. 3. This channel 2b is connected to the channel formed in the cylindrical member 2a and guides the hydrogen or oxygen gas as indicated by an arrow in FIG. 3.

Subsequently, an aromatic heater portion 32 for generating an aromatic air will be described.

First, a contact terminal 37 of the battery 36 is inserted into the upper-end opening of the battery receiving portion 43 of the body cover 1. The contact terminal 37 is formed by connecting a bottom portion of a large-diameter cylinder and an upper part of a small-diameter cylinder, the bottom portion is inserted into the opening in the upper end of the battery receiving portion 43, and power from the battery 36 is supplied to the aromatic heater portion 32. The contact terminal 37 is fastened to a joint 8 from above by cross recessed flat head screws 39. The joint 8 is formed by connecting the bottom portion and the upper part having a large diameter and a substantially disk shape of the small-diameter cylinder, and the upper part of the contact terminal 37 and the bottom portion of the joint 8 are fitted in a nested state.

The aromatic heater member 32 is placed on an upper surface of the joint 8, and when the mixer 2 described above is to be attached, it is sandwiched by the joint 8 and the mixer 2 and is fixed to the body cover 1. The aromatic heater member 32 is a general-purpose device, and when power is supplied, an air with aroma is generated therein and is emitted upward. Moreover, a cylindrical member 2c extending downward in parallel with the cylindrical member 2a described above is provided on the mixer 2, and an upper end of the aromatic heater portion 32 is connected to this cylindrical member 2c. Therefore, the air with aroma emitted from the aromatic heater portion 32 passes through the cylindrical member 2c as indicated by the arrow in FIG. 3, merges with the hydrogen or oxygen gas flowing through the channel 2b through the cylindrical member 2a, flows into the nozzle 5 and is emitted into the mouth of the user.

The nozzle 5 has a structure in which a large diameter and substantially disk-shaped member on the bottom portion and the cylindrical member on the upper part are integrally connected, and the bottom portion is attached onto the opening in the top surface fluidically connected to the cylindrical member 2c of the heater portion 32 in the mixer 2. As a result, the hydrogen or oxygen gas from the channel 2b and/or the air with aroma from the cylindrical member 2c are emitted from inside the nozzle 5 to the outside of the upper end. The O-ring 22 is disposed on the connection portion between the bottom portion of the nozzle 5 and the mixer 2 and sealed.

Moreover, the aromatic heater portion 32 controls power supply from the battery 36 by the control substrate 33. As described above, the power to the mesh electrode 17 is supplied for the predetermined time by pressing on the button 35 attached to the body cover 1 three times. On the other hand, by holding down the button, the contact terminal 37 is connected under a condition that the power supply signal to the mesh electrode 17 is not transmitted in the control substrate 33, and the power from the battery 36 is supplied to the aromatic heater portion 32 for the predetermined time.

Therefore, by pressing on the button 35 three times, when the user suctions through the nozzle 5, the hydrogen or oxygen gas is emitted from the nozzle 5, and hydrogen or oxygen gas suctioning can be enjoyed for the predetermined time (while the LED substrate 30 emits light), and by holding down the button 35 while the hydrogen or oxygen gas is emitted, the hydrogen or oxygen gas with aroma can be enjoyed.

The “configuration excluding the partition member and the opening/closing means” has been described above. Subsequently, the “configuration of the partition member and the opening/closing means” in the electrolysis tank 10 of the electrolytic hydrogen and oxygen gas suction tool of the present invention will be described by using FIGS. 4 to 6.

FIG. 4 is a view illustrating the lower part (reduced diameter portion 45) of the electrolysis tank of the electrolytic hydrogen and oxygen gas suction tool of the present invention and illustrates the reduced diameter portion 45 in the view corresponding to a sectional view along a line B-B in FIG. 2(b). In the reduced diameter portion 45, the pair of mesh electrodes are disposed by standing upright from the bottom surface, and the partition member 50 is disposed between them by separating them.

The partition member 50 is integrated with and connected to the reduced diameter portion 45 on an inner side surface in a longitudinal direction (front side and depth side in the drawing) and is a plate-shaped member extending downward from the upper part of the reduced diameter portion 45 without being connected to the bottom surface so as to divide the opening 45c. The partition member 50 is configured by the material similar to the reduced diameter portion 45 not transmitting a liquid or a gas. The opening 45c is divided into two openings, that is, an opening 45c1 and an opening 45c2 by the partition member 50, through which only hydrogen or oxygen generated from the electrodes below, respectively, passes, and moreover, it is selectively opened/closed by the opening/closing means which will be described later, whereby passage of either one of hydrogen and oxygen or both is made possible as the entire opening 45c.

In the partition member, shapes of the upper part of the reduced diameter portion 45 and the openings 45c1 and 45c2 can be configured arbitrarily, and an upper end of the partition member and the upper part of the reduced diameter portion 45 may be integrated and form a surface including two opening holes (45c1, 45c2).

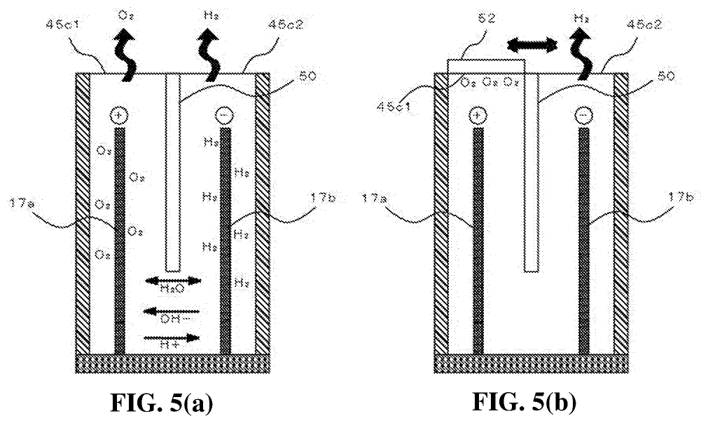

Subsequently, movement of hydrogen and oxygen in FIG. 4 will be described by using FIG. 5. In FIG. 5(a), when the electrode 17 is electrically conducted, oxygen (O₂) is generated in the vicinity of a positive electrode 17a and hydrogen (H₂) is generated in the vicinity of a negative electrode 17b. Since the generated oxygen and hydrogen have specific gravities smaller than water, they move upward and move to the openings 45c1 and 45c2, respectively. Here, since the partition member 50 is disposed between the electrodes 17a and 17b, mixing between the oxygen and hydrogen is inhibited during the upward movement of oxygen and hydrogen. On the other hand, in the lower part of the reduced diameter portion 45 not partitioned by the partition member, free movement of water (H2O), that is, movement of ions (“OH⁻” and “H⁺”) required for generation of oxygen and hydrogen is possible. As described above, the inhibition of the mixing between oxygen and hydrogen is achieved by the partition member 50 while electrolyzing.

FIG. 5(b) illustrates schematic views of movement of oxygen and hydrogen when opening/closing means 52 which will be described later is used. The opening/closing means 52 (a plate-shaped lid member in this schematic view but this is not limiting) can selectively close the opening 45c1 or 45c2 manually or electromagnetically. If the opening 45c1 is closed, for example, as illustrated in FIG. 5(b), oxygen generated in the periphery of the electrode 17a moves upward but cannot move above the opening 45c1 by the opening/closing means 52 and remains in the reduced diameter portion 45. On the other hand, hydrogen generated in the periphery of the electrode 17b moves upward from the opening 45c2 and can be suctioned in the end. By moving the opening/closing means 52 to the right direction on the drawing to above 45c2, hydrogen similarly remains in the reduced diameter portion 45, while the oxygen can move to above the opening 45c1. Moreover, by moving the opening/closing means 52 to the center between the openings 45c1 and 45c2 to the right direction on the drawing, since the openings 45c1 and 45c2 are not fully closed, both oxygen and hydrogen move upward from the opening 45.

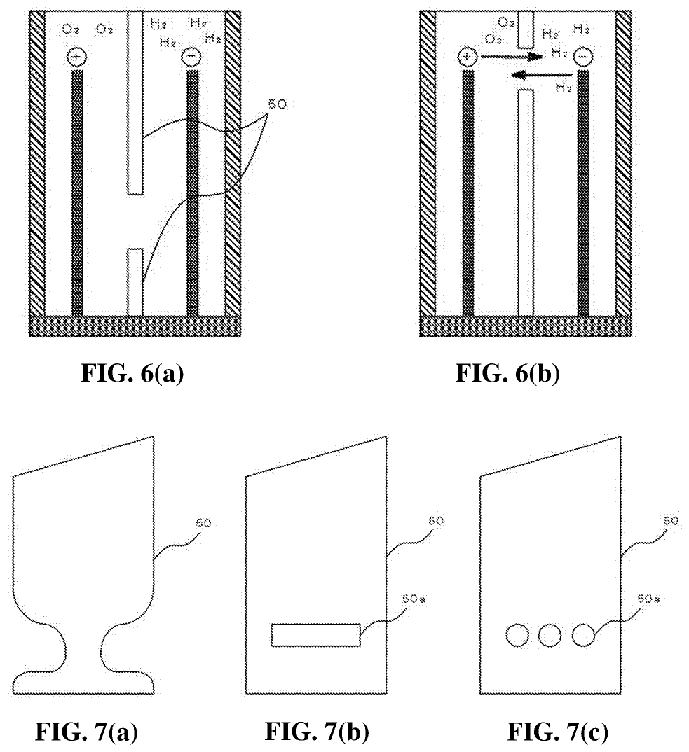

FIGS. 6 illustrate schematic views of examples of movement of hydrogen and oxygen in FIG. 4 when another partition member is employed. In FIG. 6(a), a passage through which water can pass is formed by the partition member above FIG. 4. In this example, since the passage is provided in a lower part of the reduced diameter portion 45, it is less likely that oxygen and hydrogen having moved upward pass through the passage and are mixed. In FIG. 6b, a passage is formed in an upper part of the reduced diameter portion 45 by the partition member. In this example, when amounts of oxygen and hydrogen having moved upward become large, it is more likely that they pass through the passage and are mixed. Therefore, design of the partition member so as to form a desired passage position in accordance with a use situation or a need of a degree of mixing of oxygen and hydrogen can be realized.

FIG. 7 illustrate another embodiment of the partition member 50 of the electrolytic hydrogen and oxygen gas suction tool of the present invention. FIGS. 7(a), 7(b), and 7(c) all illustrate the partition member 50 in the sectional view along the line A-A in FIG. 2(c). FIG. 7(a) illustrates the partition member 50 having inclination in an upper part similar to the upper part of the reduced diameter portion 45 and a constriction shape in a lower part, and the fluidical connection of the pair of electrodes 17 is made possible from the constriction. FIG. 7(b) illustrates the partition member 50 including the similar inclination and a substantially rectangular hole 50a in the lower part, and the fluidical connection of the pair of electrodes 17 from the hole 50a is made possible. FIG. 7(c) illustrates the partition member 50 having the similar inclination and a plurality of substantially circular holes 50a in the lower part, and the fluidical and gaseous connection of the pair of electrodes 17 from the holes 50a is made possible. The partition member 50 is not limited to the examples illustrated here, but a free shape which enables fluidical connection of the pair of electrodes 17 is employed. Moreover, FIG. 7 illustrate an example when the upper part of the reduced diameter portion 45 is inclined, but when a shape in which the upper part of the reduced diameter portion 45 is not inclined or any other shape is employed, the upper part of the partition member 50 also has the shape similar to the upper part of the reduced diameter portion 45.

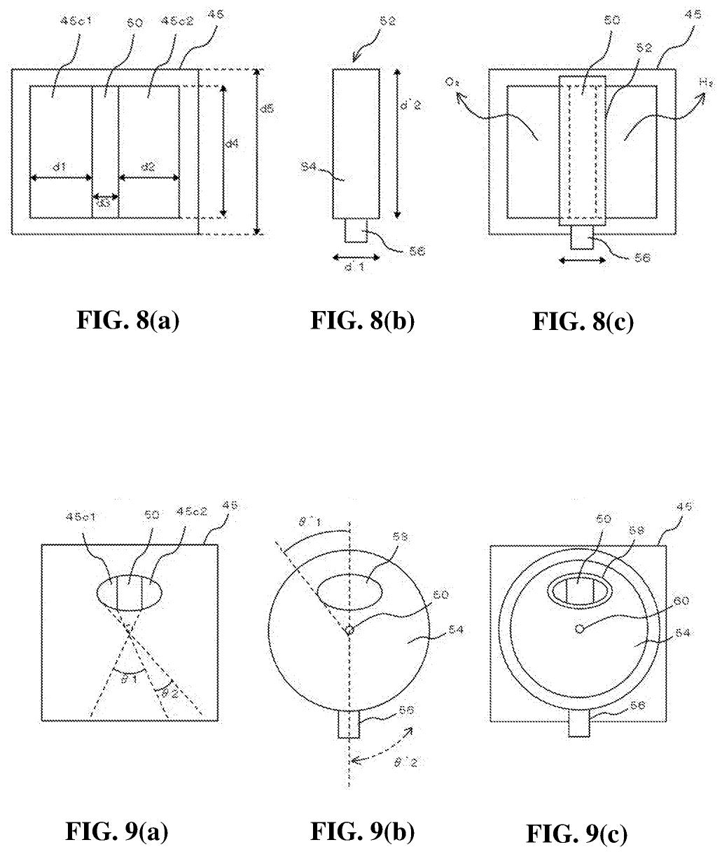

Subsequently, the opening/closing means 52 for selectively obtaining hydrogen and oxygen will be described by using FIGS. 8 to 10. In the example of the electrolytic hydrogen and oxygen gas suction tool in FIGS. 1 to 3, the water shielding plate 45d is disposed in the reduced diameter portion 45 and the opening 45c is inclined, but these configurations do not have to be included, and in FIGS. 8, an example in which the water shielding plate 45d is not included, and the opening 45c is not inclined either will be described.

FIG. 8(a) illustrates a top view of the opening of the lower part (reduced diameter portion 45) of the electrolysis tank of the electrolytic hydrogen and oxygen gas suction tool of the present invention. As described above, the opening 45c is divided into the openings 45c1 and 45c2 by the partition member 50, and each has a substantially rectangular opening shape. Here, assume that lengths of the openings 45c1 and 45c2 and the partition member 50 in the lateral direction on the drawing are d1, d2, and d3, respectively, and the lengths of the openings 45c1 (45c2) and the reduced diameter portion 45 in the vertical direction on the drawing are d4 and d5, respectively. FIG. 8(b) illustrates the opening/closing means 52, and the opening/closing means 52 is constituted by connecting an operation switch 56 to one side of a substantially rectangular shielding portion 54. By causing the shielding portion 54 to abut to the opening 45c1 or 45c2 while air tightness (water tightness) is provided by an O-ring or the like, passage of oxygen or hydrogen from each of the openings can be prevented. Here, a length of a side where the operation switch 56 of the shielding portion 54 is connected is assumed to be d′1, and a length of a side adjacent to that to be d′2.

The distance d′1 of the opening/closing means 52 satisfies d′1<d1+d2+d3, d′1>d1+d3, and d′1>d2+d3, and the distance d′2 satisfies d′2<d5 and d′2>d4. By means of this configuration, selection of hydrogen and oxygen is made possible as illustrated in FIG. 8(c). FIG. 8(c) is a view in which the opening/closing means 52 in FIG. 8(b) is attached to the opening (45c1, 45c2) in FIG. 8(a) from above, and the operation switch 56 protrudes toward an outside of the reduced diameter portion 45, and by sliding the operation switch 56 in the lateral direction on the drawing, the shielding portion 54 can be slid in the lateral direction. The example of FIG. 8(c) illustrates an example in which the opening/closing means 52 is located above the partition member 50, and in this case, since neither the openings 45c1 nor 45c2 is fully closed by the shielding portion 54, both hydrogen and oxygen generated from the electrode 17 pass through the openings 45c1 and 45c2 and move toward above the reduced diameter portion 45 (upper part of the electrolysis tank). Here, by sliding the operation switch 56 to the left direction on the drawing and by closing the opening 45c1 by the shielding portion 54, only hydrogen (oxygen) moves to the upper part of the electrolysis tank. To the contrary, by sliding the operation switch 56 to the right direction on the drawing, and by closing the opening 45c2 by the shielding portion 54, only oxygen (hydrogen) moves to the upper part of the electrolysis tank. The operation switch 56 can be slid within a range in which the shielding portion 54 is in the reduced diameter portion 45.

FIG. 9(a) is a top view of the opening of the lower part (reduced diameter portion 45) of the electrolysis tank of the electrolytic hydrogen and oxygen gas suction tool of the present invention and illustrates another example of the opening/closing means 52. An upper end of the reduced diameter portion 45 forms an upper surface including the substantially oval opening 45, and the opening 45 is divided into the openings 45c1 and 45c2 by the partition member 50. The opening 45 is disposed on the drawing above the center of the upper surface of the reduced diameter portion 45. Here, assume that an angle formed by a straight line connecting the center of the reduced diameter portion 45 and a left end on the drawing of the opening 45c1 and a straight line connecting the center of the reduced diameter portion 45 and the left end on the drawing of the partition member 50 is θ1, and an angle formed by a straight line connecting the center of the reduced diameter portion 45 and the left end on the drawing of the partition member 50 and a straight line connecting the center of the reduced diameter portion 45 and a right end on the drawing of the partition member 50 is θ2.

FIG. 9(b) illustrates another example of the opening/closing means 52, and the opening/closing means 52 is constituted by connecting the operation switch 56 to an outer periphery of the substantially circular shielding portion 54 having a substantially oval opening 58. A rotating shaft 60 is disposed at a center of the shielding portion 54, and the operation switch 56 is disposed on the outer periphery of the shielding portion 54 by facing the opening 58 through the rotating shaft 60. The opening 58 has an opening width larger than a width of the partition member 50. By causing the shielding portion 54 to abut to the opening 45c1 or 45c2 while providing air tightness (water tightness) by the O-ring (not shown), passage of oxygen or hydrogen from each of the openings can be prevented. Here, assume that an angle formed by a straight line connecting the rotating shaft and a left end (right end) on the drawing of the opening 58 and a straight line connecting the rotating shaft and the operation switch 56 is θ′1. Moreover, by causing the operation switch to move along a curved line projecting downward to right and left on the drawing, the shielding portion 54 can be rotated around the rotating shaft 60. An angle of this curved movement (rotary movement) of the operation switch is assumed to be θ′2.

FIG. 9(c) is a view in which the opening/closing means 52 in FIG. 9(b) is attached to the opening (45c1, 45c2) in FIG. 9(a) from above, and the operation switch 56 protrudes to the outside of the reduced diameter portion 45, and by rotating the operation switch 56, the shielding portion 54 can be rotated with respect to the rotating shaft 60. The example in FIG. 9(c) illustrates an example in which the opening 58 of the opening/closing means 52 is located above the partition member 50, and in this case, since neither the opening 45c1 nor 45c2 is fully closed by the shielding portion 54, hydrogen and oxygen generated from the electrode 17 pass through the openings 45c1 and 45c2 and move toward above the reduced diameter portion 45 (upper part of the electrolysis tank). Here, by rotating the operation switch 56 to the right direction on the drawing and by closing the opening 45c2 by the shielding portion 54, only oxygen (hydrogen) moves to the upper part of the electrolysis tank. To the contrary, by rotating the operation switch 56 to the right direction on the drawing and by closing the opening 45c1 by the shielding portion 54, only hydrogen (oxygen) moves to the upper part of the electrolysis tank. The operation switch 56 can rotate at least either one of the opening 45c1 and 45c2 within a range in which the shielding portion 54 is closed, the opening 58 is a symmetric oval, and when the partition member 50 is disposed at the center of the opening 58 so as to be seen through the opening 58, the operation switch at a position faced with the partition member 50 through the rotating shaft can be made to slide to right and left within a range of θ′2>θ′1+θ2 and θ′2<θ′1+θ1+θ2.

Assuming the use situation of the electrolytic hydrogen and oxygen gas suction tool of the present invention, setting that the opening 45c1 (45c2) on the oxygen side is closed (state in which the operation switch 56 is slid to the left in the case of FIG. 8(c)) may be initial setting so that only hydrogen with a higher use frequency can pass.

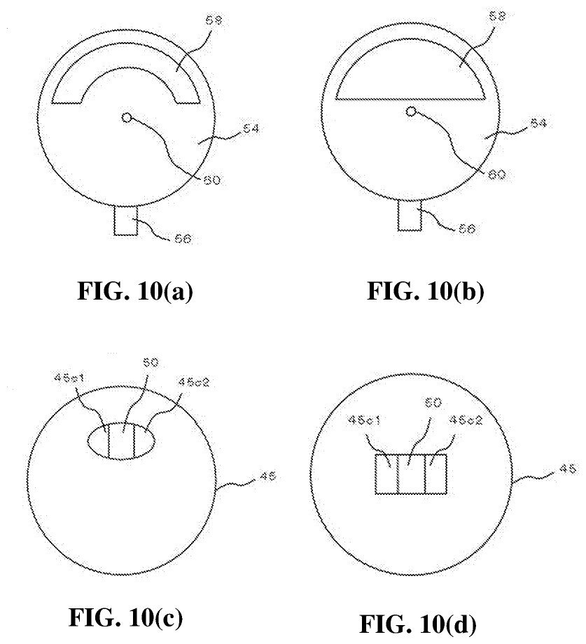

Subsequently, by using FIGS. 10, other examples of the opening/closing means 52 and the upper part (upper surface) of the reduced diameter portion 45 of the electrolytic hydrogen and oxygen gas suction tool of the present invention will be described. FIGS. 10(a) and 10(b) are other examples of the opening/closing means 52 and have configuration in which the operation switch 56 is rotated/moved as in FIG. 9(b). The opening/closing means 52 illustrated in FIG. 10(a) has a band shape curved so that the opening 58 surrounds the rotating shaft 60. The opening/closing means 52 illustrated in FIG. 10(b) has the opening 58 with a substantially semicircular shape. And FIGS. 10(c) and 10(d) are other examples of the upper part (upper surface) of the reduced diameter portion 45. FIG. 10(c) illustrates that the upper part (upper surface) of the reduced diameter portion 45 is a substantially circular surface, the substantially oval opening 45c is disposed out of the center on the surface, and the opening 45c is divided by the partition member 50 into the openings 45c1 and 45c2. This configuration can be employed together with the opening/closing means 52 having configuration in which the operation switch 56 in FIG. 9(b) or the like is rotated/moved. FIG. 10(d) illustrates that the upper part (upper surface) of the reduced diameter portion 45 is a substantially circular surface, the substantially rectangular opening 45c is disposed substantially at a center on the surface, and the opening 45c is divided by the partition member 50 into the openings 45c1 and 45c2. This configuration can be employed together with the opening/closing means 52 having configuration in which the operation switch 56 in FIG. 8(b) or the like is laterally moved.

As described above, the opening/closing means 52, the upper part of the reduced diameter portion 45, and the opening 45 have been described, but it is only necessary that the two openings 45c1 and 45c2 can be selectively opened/closed other than the examples illustrated in the figures and description, and shapes and locations of the opening of 58 in the opening/closing means 52, the shape and mechanism of the operation switch 56, the shape of the upper part of the reduced diameter portion 45, and the shape and location of the opening 45 can be widely generalized and employed.

Moreover, the operation switch 56 protrudes toward the outside (side surface of the body) of the reduced diameter portion 45 in the examples in FIGS. 8 to 10, and the manual operation by the user is assumed, but it may be so configured that the operation switch is not provided but a switch operation is made electromagnetically through the control plate. At that time, a stepping motor or the like can be employed. Moreover, not the configuration in which the plate-shaped opening/closing means 52 as illustrated in FIGS. 8 to 10 is planarly moved with respect to the opening 45 but configuration in which a door is opened/closed with respect to the opening 45 (electromagnetic valve, for example) may be employed. Other than the above, a biasing force toward an initial setting position of opening/closing selection may be provided in the opening/closing means, and the setting of opening/closing may be switched by applying a force against the biasing force by an operation.

Moreover, for the hydrogen and oxygen gas suction tool of the second invention, configuration having the polarity inverting means (polarity inversion circuit) by which hydrogen or oxygen can be taken in without using the opening/closing means can be considered. Exemplification/description will be made below.

More specifically, an embodiment of the hydrogen and oxygen gas suction tool of the second invention is illustrated as a variation in FIGS. 4 to 5, and FIGS. 12 to 13 illustrate examples of the polarity inversion circuit.

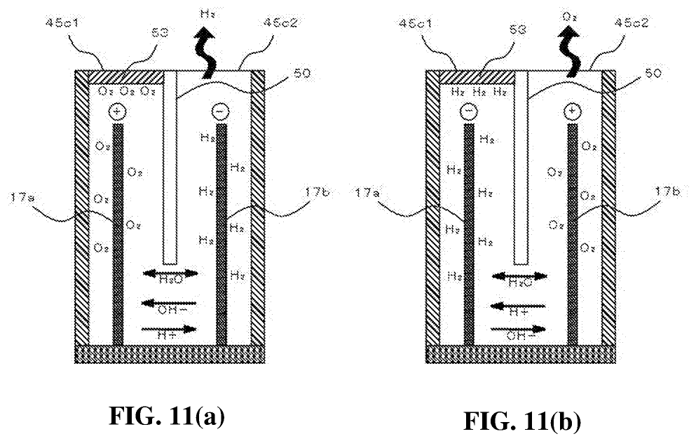

As illustrated in FIG. 11, one of the opening 45c1 and the opening 45c2 on the upper part of the reduced diameter portion 45 in the electrolysis tank 10 is closed by a closing plate 53 to the upper end of the partition plate 50 so as not to be connected in a gaseous manner to the upper part of the electrolysis tank 10. In the state in FIG. 11(a), the electrode 17a is a positive electrode, and a state in which oxygen is generated from the positive electrode 17a, collects on the lower surface of the closing plate 53 and is not emitted upward is illustrated. On the other hand, the electrode 17b is a negative electrode, and a state in which hydrogen is generated from the negative electrode 17b and is emitted upward from the opening 45c2 is illustrated.

Moreover, FIG. 11(b) illustrates a state in which the polarities of the electrodes 17a and 17b are inverted from FIG. 11(a) by the polarity inversion circuit which will be described later. That is, the electrode 17a has been inverted to the negative electrode, and hydrogen is generated from the negative electrode 17a and collects on the lower surface of the closing plate 53. On the other hand, oxygen is generated from the positive electrode 17b and is emitted upward from the opening 45c2.

As described above, when the polarities of the electrodes 17a and 17b are inverted by the polarity inversion circuit, hydrogen and oxygen generated from each of the electrodes 17a and 17b are switched, and by keeping on closing either of the openings 45c1 and 45c2, the gas emitted to above the electrolysis tank 10 is also either one of hydrogen or oxygen.

The change from FIGS. 11(a) to 11(b) is effected by the polarity inversion circuit, but immediately after the polarity is inverted, since time is needed for hydrogen generation and oxygen generation to be switched, short time exists during which a gas is not generated from the both electrodes 17a and 17b. As a result, the polarity inversion circuit can be also utilized as a role of a brake for the hydrogen generation or the oxygen generation, and generation amounts/generation time of hydrogen or oxygen can be controlled precisely. Therefore, it is advantageous to use the polarity inversion circuit at the same time even when the aforementioned opening/closing means 52 is used.

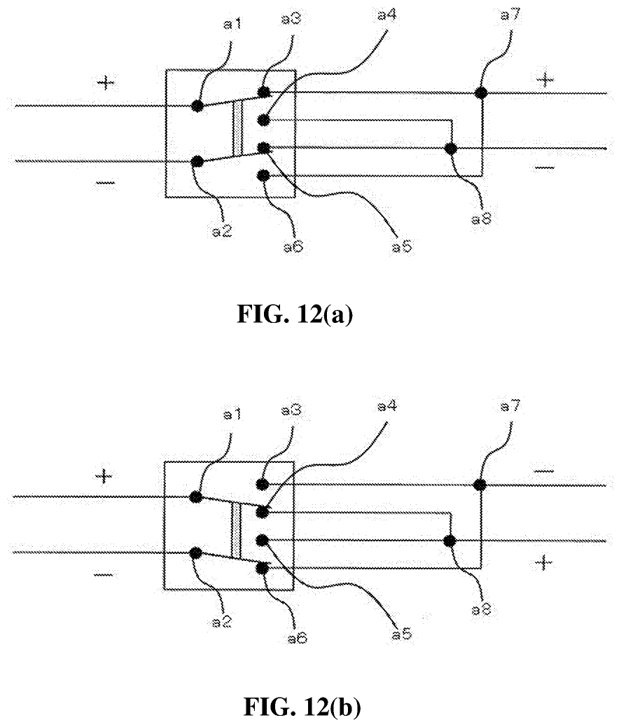

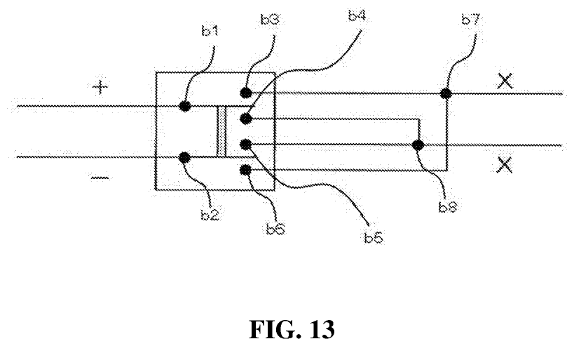

Subsequently, the polarity inversion circuit will be described. FIGS. 12 to 13 exemplify two polarity inversion circuits. There can be cases for the polarity inversion circuit in which the control substrates 33 and 42 (see FIG. 1) directly or indirectly conducting power supply to the electrodes 17 (17a, 17b) has the polarity inversion circuit or it is connected separately in the middle between the control substrates 33 and 42 to the electrodes 17.

The polarity inversion circuit in FIGS. 12(a) and 12(b) is a method in which the polarity is switched each time the alternate-type switch is turned ON, and FIG. 12(a) illustrates a state before the polarity inversion, and FIG. 12(b) after the polarity inversion. As described above, the alternate-type switch is a method in which the ON state is held even after the hand leaves the button after pressing it, and in this case, once the button is pressed to be turned ON, hydrogen or oxygen is continuously emitted, and when the button is pressed again, oxygen can be emitted.

More specifically, in the state in FIG. 12(a), a contact al which is a positive electrode is connected to a contact a3, while a contact a2 which is a negative electrode is connected to a contact a5, and contacts a1-a3-a7- and after (right side in the figure) are positive electrodes, and contacts a2-a5-a8 - and after (right side in the figure) are negative electrodes. When the polarity is inverted from this state and the switch is operated, the state is switched to that in FIG. 12(b), and the contact al which is the positive electrode is connected to the contact a4, and the contact a2 which is the negative electrode to the contact a6. As a result, the polarities are inverted to such that the contacts a1-a4-a8- and after (right side in the figure) are inverted to positive electrodes and the contacts a2-a6-a7- and after (right side in the figure) are inverted to negative electrodes.

Moreover, FIG. 13 is the polarity inversion circuit which switches the polarity by switching like ON-OFF-ON. With this polarity inverting means, in the case of OFF, the contact bl which is a positive electrode is between the contacts b3-b4 and the contact b2 which is the negative electrode is located between the contacts b5-b6, which is a non-contact state. When the switch is operated to ON and the contacts b1 and b2 are connected to the contacts b3 and b5, respectively, the contact b1 which is a positive electrode is connected to the contacts b3-b7- and after (right side in the figure), and the contact b2 which is a negative electrode is connected to the contact b5-b8- and after (right side in the figure). Then, when the switch is turned OFF and then, operated to ON, the contacts bl and b2 are connected to the contacts b4 and b6, respectively, the contact bl which is a positive electrode is connected to the contacts b4-b8- and after (right side in the figure), and the contact b2 which is a negative electrode is connected to the contacts b6-b7- and after (right side in the figure), in which the polarity is inverted.

The switch of the polarity inversion circuit in FIG. 13 may be of the alternate-type as in FIGS. 11 to 12, but a momentary type in which ON is kept only while the button is pressed or concomitant use of the momentary type and the alternate type may be employed. Assuming that ON=the alternate type and (ON)=the momentary type, (1) ON-OFF-ON, (2) ON-OFF-(ON), and (3) (ON)-OFF-(ON) can be considered.

The embodiments of the hydrogen and oxygen gas suction tool of the present invention have been exemplified/described, but the present invention is not limited to them, and those skilled in the art can understand that other modifications and improvement examples can be obtained within a range not departing from the sprit or teaching described in the appended claims and description and the like.

### INDUSTRIAL APPLICABILITY

According to the electrolytic hydrogen and oxygen gas suction tool of the present invention, in a charging type so that the user can freely carry, though the battery is small-sized and inexpensive, a space for incorporating the battery is ensured, water shielding between the electrolysis tank and the battery is ensured, and moreover, a sufficient amount of hydrogen gas generated can be ensured even if it is inclined in a state where a moisture in the electrolysis tank is decreased.

### REFERENCE SIGNS LIST

100 electrolytic hydrogen and oxygen gas suction tool

1 body cover

2 mixer

13 hydrogen passing member

13a film material (breathable impermeable material)

14 ample portion

15 lid member

16 metal material

17 container body portion

18 aqueous solution

19 closing member

20 hydrogen

22 non-reaction portion

24 metal particle layer

40 projecting shaped portion

41 thin portion

50 partition member

52 opening/closing means

53 closing plate

54 shielding portion

56 operation switch

58 opening

60 rotating shaft

100, 200 hydrogen and oxygen gas suction tool

102 suction tool body portion

104 suctioning sheath portion

105 cap member

106, 206 connection portion

108, 208 mouth member

110, 210 film packing

112 control valve

113, 213 window

114 adjustment port

116 cartridge

117, 217 gap

118 O-ring

1. An electrolytic hydrogen and oxygen gas suction tool, comprising:
an electrolysis tank capable of storing water and constituted by an upper part and a lower part which are fluidically connected to each other therein and integrally molded;
a pair of electrodes disposed in the lower part in the electrolysis tank, standing substantially in parallel with a vertical direction of the electrolysis tank, and faced with each other in a lateral direction;
a battery; and
a control substrate which supplies power from the battery, wherein
the electrode is supplied with or shut off from power supply from the battery by the control substrate;
a partition member extending downward from a boundary between the upper part and the lower part of the electrolysis tank passing between the pair of electrodes is integrally molded and provided in the lower part of the electrolysis tank;
the pair of electrodes are fluidically connected to each other in the lower part of the electrolysis tank; and
opening/closing means which enables switching of gaseous connection between one and/or the other of the pair of electrodes separated by the partition member and the upper part of the electrolysis tank is provided.

2. The electrolytic hydrogen and oxygen gas suction tool according to claim 1, wherein
in the lower part of the electrolysis tank and above the pair of electrodes, the partition member is formed by a plate member, and passage of a fluid and a gas is shut off between one surface side and the other surface side of the partition member.

3. The electrolytic hydrogen and oxygen gas suction tool according to claim 1, wherein
the opening/closing means is a member provided on a boundary between the upper part and the lower part of the electrolysis tank and having a closed and substantially flat area, and the substantially flat area moves on a substantially plane in parallel with the boundary between the upper part and the lower part of the electrolysis tank in accordance with an operation by a user.

4. The electrolytic hydrogen and oxygen gas suction tool according to claim 1, wherein
the battery is disposed in parallel in the vertical direction of the electrolysis tank;
above the battery, an aromatic gas generating member in which on/off control of aromatic gas generation is conducted by the control plate is disposed; and
a channel in which the aromatic gas is merged with the gas emitted from the electrolysis tank is provided.

5. The electrolytic hydrogen and oxygen gas suction tool according to claim 1, wherein
the opening/closing means is controlled by the control plate.

6. The electrolytic hydrogen and oxygen gas suction tool according to claim 1, wherein
the opening/closing means includes a plate-shaped shielding portion on the boundary with the lower part of the electrolysis tank; and
gaseous connection between one and/or the other of the pair of electrodes and the upper part of the electrolysis tank is made switchable by parallel and/or rotary movement of the shielding portion on the boundary.

7. An electrolytic hydrogen and oxygen gas suction tool, comprising:
an electrolysis tank capable of storing water and constituted by an upper part and a lower part which are fluidically connected to each other therein and integrally molded;
a pair of electrodes disposed in the lower part in the electrolysis tank, standing substantially in parallel with a vertical direction of the electrolysis tank, and faced with each other in a lateral direction;
a battery; and
a control substrate which supplies power from the battery, wherein
the electrode is supplied with or shut off from power supply from the battery by the control substrate;
a partition member extending downward from a boundary between the upper part and the lower part of the electrolysis tank passing between the pair of electrodes is integrally molded and provided in the lower part of the electrolysis tank;
the pair of electrodes are fluidically connected to each other in the lower part of the electrolysis tank;
gaseous connection between one side of the pair of electrodes separated by the partition member and the upper part of the electrolysis tank is shut off and the gaseous connection between the other side of the electrode and the upper part of the electrolysis tank is opened; and
polarity inverting means which inverts polarity of power supplied to each of the pair of electrodes is provided.

8. The electrolytic hydrogen and oxygen gas suction tool according to claim 7, wherein
the polarity inverting means has a polarity circuit which switches polarity of power supplied from the battery each time an alternate-type switch is turned ON.

9. The electrolytic hydrogen and oxygen gas suction tool according to claim 7, wherein
the polarity inverting means has a polarity circuit which switches polarity of power supplied from the battery by turning ON-OFF-ON a switch.

10. The electrolytic hydrogen and oxygen gas suction tool according to claim 9, wherein the switch is of a momentary type.

11. The electrolytic hydrogen and oxygen gas suction tool according to claim 1, further comprising:
polarity inverting means which inverts polarity of power supplied to each of the pair of electrodes.
